(19) United States
(12) Plant Patent Application Publication
(10) Pub. No.: US 2017/0020046 P1
(43) Pub. Date: Jan. 19, 2017

(54) Strawberry plant named 'Drisstrawfortyseven'

(50) ;

(71) Applicant: DRISCOLL STRAWBERRY ASSOCIATES, INC., WATSONVILLE, CA (US)

(72) Inventors: Michael D. Ferguson, Moorpark, CA (US); Terrance C. Moran, Naples, FL (US); Josefa Lagunas, Port Hueneme, CA (US)

(21) Appl. No.: 14/545,992

(22) Filed: Jul. 15, 2015

(51) Int. Cl. A01H 5/00

(52) U.S. Cl. PLT/208

(57) ABSTRACT

A new and distinct variety of strawberry plant named ‘DrisStrawFortySeven’ particularly characterized by having large, dark red, sweet fruit, a globose semi-upright plant habit, and resistance to Tetranychus urticae, is disclosed.

### GENUS AND SPECIES

Fragaria×ananassa

### VARIETY DENOMINATION

‘DrisStrawFortySeven’

### BACKGROUND OF THE NEW PLANT

The present invention relates to a new and distinct strawberry variety designated ‘DrisStrawFortySeven’ and botanically known as Fragaria×ananassa. This new strawberry variety was discovered in Ventura County, California in January 2009 and originated from a cross between the proprietary female parent ‘41Q324’ (unpatented) and the proprietary male parent ‘DrisStrawThirtySeven’ (U.S. patent application Ser. No. 13/998,836). A single plant was selected and asexually propagated via tissue culture and vegetative cuttings in Shasta County, California in 2009.

‘DrisStrawFortySeven’ underwent further testing in Ventura County, California from 2010-2015. The present invention has been found to retain its distinctive characteristics through successive asexual propagations via stolons and tissue culture.

Plant Breeder's Rights for this variety have not been applied for. ‘DrisStrawFortySeven’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Ventura County, California.

1. Large, dark red, sweet fruit;

2. Globose semi-upright plant habit; and

3. Resistant to Tetranychus urticae.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from five-month-old plants.

FIG. 1 shows upper and lower surfaces of the leaves of the plant with three leaflets.

FIG. 2 shows both upper and lower surfaces of the flowers.

FIG. 3 shows the whole fruit.

FIG. 4 shows the fruit in longitudinal cross-section.

FIG. 5 shows the entire plant habit.

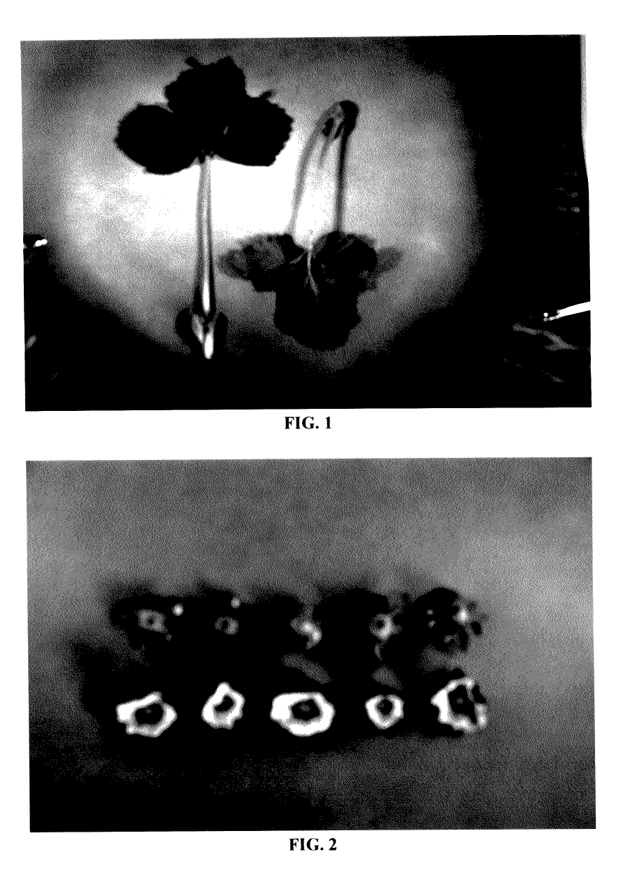

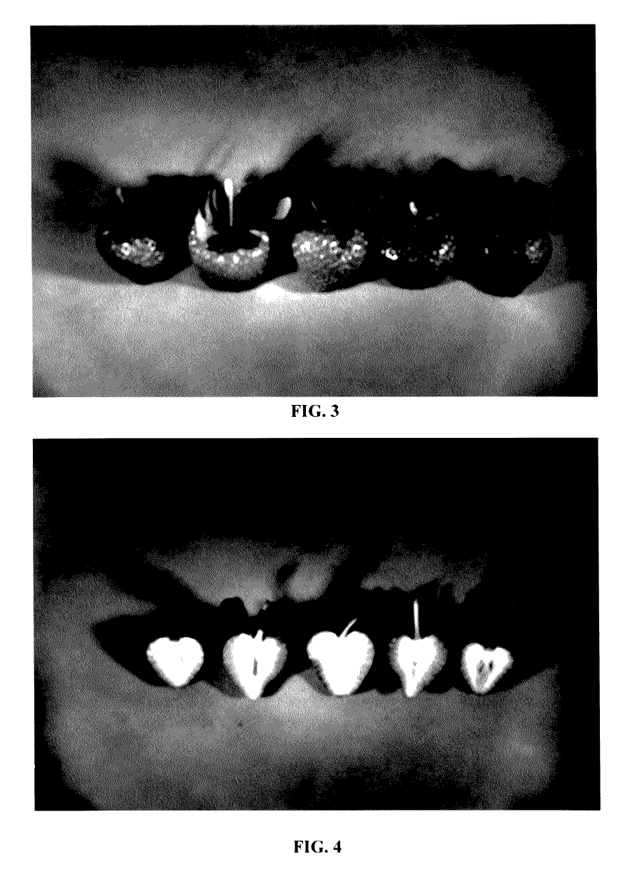

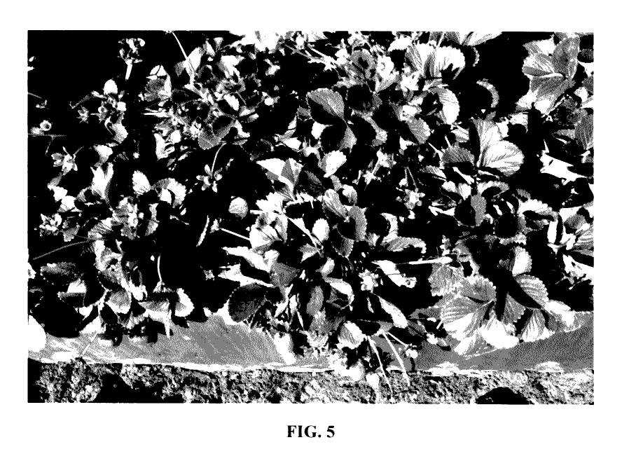

### DESCRIPTION OF THE NEW VARIETY

The following detailed descriptions set forth the distinctive characteristics of ‘DrisStrawFortySeven’. The data which define these characteristics is based on observations taken in Ventura County, California from 2010 to 2015. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFortySeven’ has not been observed under all possible environmental conditions. The botanical description of ‘DrisStrawFortySeven’ was taken from five-month-old plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria×ananassa.
   Common name.—Strawberry.
   Denomination.—‘DrisStrawFortySeven’.
Parentage:
   Female parent.—The proprietary variety ‘41Q324’ (unpatented).
   Male parent.—The proprietary variety ‘DrisStrawThirtySeven’ (U.S. patent application Ser. No. 13/998,836).
Plant:
   Height.—20.5 cm.
   Diameter.—34.8 cm.
   Number of crowns/plant.—4.
   Habit.—Globose — semi-upright.
   Density of individual plant.—Medium.
   Vigor (health and hardiness of plant).—Medium.
Terminal leaflets:
   Size.—Medium Length: 7.2 cm Width: 6.5 cm Length/width ratio: 1.1 (Longer than broad).
   Number of teeth/terminal leaflet.—23.
   Shape of teeth.—Rounded — crenate.
   Color.—Upper surface: RHS N137A (Dark green) Lower surface: RHS 147C (Medium yellow-green).
   Shape in cross section.—Concave.
   Blistering.—Weak.
   Glossiness.—Medium.
   Number of leaflets.—Three only.
   Shape.—Orbicular.
   Base shape.—Rounded.
   Apex descriptor.—Rounded.
   Margin.—Crenate.
   Margin profile.—Both revolute (margins rolled backwards) and flat (level with the leaflet blade).
   Variegation.—Absent.
Petiole:
   Length.—Medium; 12.2 cm.
   Diameter.—4.32 mm.
   Pubescence.—Dense.
   Pose of hairs.—Slightly upwards.
   Color.—RHS 144D (Light yellow-green).
   Bract frequency.—0.
Petiolule:
   Length.—7.20 mm.
   Diameter.—2.07 mm.
   Color.—RHS 144D (Light yellow-green).
Stipule:
   Length.—3.9 cm.
   Width.—10.17 mm.
   Pubescence.—Dense.
   Stipule anthocyanin coloration.—Medium; RHS 63D (Light red-purple).
Inflorescence:
   Position relative to foliage.—Above.
   Number of flowers per plant.—Many; 18.70.
   Time of flowering (50% of plants at first flower).—Early; late November — early January.
   Flower size.—Medium.
   Flower diameter.—23.10 mm.
   Petals.—Shape: Orbicular Apex: Rounded Base: Concavo-convex Margin: Entire Spacing: Overlapping Length: 16.23 mm Width: 16.70 mm Length/width ratio: 1.0 (Broader than long) Petal number per flower: 6 Color (upper surface): RHS NN155B (White).
   Calyx.—Diameter: 35.13 mm Diameter relative to corolla: Larger Inner calyx diameter relative to outer: Both smaller and same size Insertion of calyx: Level Pose of calyx segments: Both spreading — outwards and reflexed — upwards Size of calyx in relation to fruit: Slightly larger Adherence of calyx: Strong.
   Sepal.—Shape: Elliptical Apex: Convex Margin: Entire Length: 38.11 mm Width: 9.32 mm Sepal number: 12.
   Receptacle color.—RHS 151 C (Light green-yellow).
   Stamen.—Present Anther color: RHS 163C (Greyed orange).
   Pedicel.—Attitude of hairs: Upwards.
Fruiting truss:
   Length.—Long; 23.8 cm.
   Diameter at base of truss.—4.71 mm.
   Number of berries per fruiting truss.—2.
   Attitude at first picking.—Semi-erect.
   Color at base of truss.—RHS 144C (Light yellow-green).
Fruit:
   Relative fruit size.—Large.
   Length.—43.15 mm.
   Width.—44.16 mm.
   Length/width ratio.—1.0 (Broader than long).
   Fruit hollow length.—22.49 mm.
   Fruit hollow width.—21.29 mm.
   Fruit hollow length/width ratio.—1.1 (Longer than broad).
   Fruit hollow center (cavity).—Small.
   Fruit weight.—32.9 g.
   Predominant fruit shape.—Conical.
   Difference in shape between primary and secondary fruits.—Moderate.
   Evenness of fruit surface.—Slightly uneven.
   Fruit skin color.—RHS 46A (Dark red).
   Evenness of fruit color.—Even or very slightly uneven.
   Fruit glossiness.—Medium.
   Achenes.—Insertion of achenes: Level with surface Coloration (sunward side of berry): RHS 181C (Dark red) Coloration (shaded side of berry): RHS 144D (Light yellow-green) Number per berry: 199 Weight (weight of achenes divided by total # seed): 0.153 g Width of band without achenes: Narrow.
   Firmness of flesh (when fully ripe).—Firm.
   Color of flesh (excluding core).—RHS 43A (Medium red) and 69D (Light red-purple).
   Color of core.—RHS 38A (Light red).
   Evenness of flesh color.—Slightly uneven.
   Distribution of flesh color.—Marginal and central.
   Sweetness.—Strong.
   Acidity.—Weak.
   Texture when tasted.—Coarse.
   Type of bearing.—Not everbearing — not remontant.
   Harvest interval.—Mid-December — mid-January.
   Harvest maturity.—Mid-season.
   Production.—1058 grams per plant.
Disease and pest resistance:
   Tetranychus urticae.—Resistant.
   Lygus hesperus (Lygus bug).—Moderately susceptible.
   Botrytis fruit rot.—Moderately susceptible.
   Powdery mildew.—Moderately resistant.
Stress resistance:
   High temperatures.—Moderately resistant.
   Wind.—Moderately resistant.
   High pH.—Moderately resistant.
   High soil salt levels.—Moderately resistant.
   Water logging.—Moderately resistant.

### COMPARISON WITH PARENTAL AND COMMERCIAL VARIETIES

When ‘DrisStrawFortySeven’ is compared to the female parent ‘41Q324’ (unpatented), ‘DrisStrawFortySeven’ is sweeter and firmer with a more conic shape and better fruit appearance than ‘41Q324’.

When ‘DrisStrawFortySeven’ is compared to the male parent ‘DrisStrawThirtySeven’ (U.S. patent application Ser. No. 13/998,836), ‘DrisStrawFortySeven’ is sweeter and less firm with lower yields and earlier production than ‘DrisStrawThirtySeven’.

‘DrisStrawFortySeven’ differs from the commercial variety ‘DrisStrawTwentySeven’ (U.S. Plant Pat. No. 23.400), in that ‘DrisStrawFortySeven’ has a globose—semi-upright plant habit and four crown per plant, whereas ‘DrisStrawTwentySeven’ has a flat globose plant habit and three crowns per plant. Additionally, ‘DrisStrawFortySeven’ has large, medium glossy fruit, whereas ‘DrisStrawTwentySeven’ has very large, strongly glossy fruit.

We claim:

1. A new and distinct variety of strawberry plant named ‘DrisStrawFortySeven’, substantially as illustrated and described herein.
